(19) United States
(12) Patent Application Publication
(10) Pub. No.: US 2011/0098620 A1
(43) Pub. Date: Apr. 28, 2011

(54) MEDICAL DEVICE FOR SKIN WITH ULTRA-HYDROPHILIC PRESSURE-SENSITIVE ADHESIVE

(75) Inventor: Gerald R. Dever, Cordova, TN (US)

(73) Assignee: Schering-Plough Healthcare Products, Inc.

(21) Appl. No.: 12/446,933

(22) PCT Filed: Dec. 18, 2008
(86) PCT No.: PCT/US08/87317

§ 371 Date: Dec. 17, 2010

Related U.S. Application Data
(60) Provisional application No. 61/015,900, filed on Dec. 21, 2007.

(51) Int. Cl. A61F 13/02; A61L 15/44

(52) U.S. Cl. 602/44; 602/54; 602/48; 602/46

(57) ABSTRACT

A cushioning device for use on skin, such as on a finger or foot, comprising a cushioning layer having a skin side and an outer side and an ultra-hydrophilic pressure-sensitive adhesive layer coupled to the skin side of the cushioning layer.

### FIELD OF THE INVENTION

Some example embodiments of the present invention are generally directed to a medical device, for application to a finger or foot, which include an ultra-hydrophilic pressure-sensitive adhesive on the skin side of the device, e.g., various types of cushions or medicated plasters.

### BACKGROUND

The wear performance of small devices or articles depends on a number of factors. Those factors include: i) the site of attachment of the device on the body; ii) the area of the surface contact between the adhesive and the skin; iii) the frictional coefficient of the device's outer surface; iv) the conformability of the device around irregularly shaped skin surface; and (v) the breathability of the device to transepidermal water from the skin, among others.

The wear performance of small devices or articles on some special body parts, for example, toes, plantar surface on the bottom of the feet, curved bunion area near a great toe, etc., may be difficult to be achieved due to the irregularly shaped skin surface, broad range of skin type, and the moist environment around the skin, e.g., the particularly humid environment of a foot inside a shoe and/or sock.

When it is time to change the devices, it is important that the device separates gently enough to avoid damage to the site of healing of the skin. Also, the adhesive should be removed cleanly from the skin with minimum adhesive residue, which can trap lint, dirt or microbes near the site.

### SUMMARY

The inventors of the present application have identified a need for improved adhesives to devices intended for use on the finger or foot. In particular, they have recognized that it would be desirable to provide an economic and well performing device, in which an effective hydrophilic pressure-sensitive adhesive provides a high-moisture vapor-transmission rate (MVTR), and, at the same time, provides a high capacity for absorption of moisture from the skin, which promotes skin adhesion and extended wear time by reducing hydration and attendant weakening of the outer-most layer of the skin (stratum corneum).

In certain example embodiments, the invention provides a medical cushioning device for use on a finger, hand, toe or foot, comprising a cushioning layer having a skin side and an outer side; and an ultra-hydrophilic pressure-sensitive adhesive layer coupled to the skin side of the cushioning layer. In certain embodiments, the ultra-hydrophilic pressure-sensitive adhesive layer may be in direct contact with the cushioning layer. In certain embodiments, the ultrahydrophilic PSA has a MVTR of at least about 1500 gram/m²/24 hours, at least about 2000 gram/m²/24 hours, at least about 2500 gram/m²/24 hours, or at least about 3000 gram/m²/24 hours. Further, in certain embodiments the ultrahydrophilic PSA has a percent water uptake of at least about 1%, at least about 1.5%, at least about 2% and at least about 2.5% (all percentages by weight of the adhesive). In certain embodiments, the ultrahydrophilic PSA has a MVTR at least about 1500 gram/m²/24 hours and moisture uptake capacity of the adhesive layer of the device of at least about 1%, at least about 1.5%, at least about 2% or at least about 2.5% (all percentages by weight of the adhesive). In certain embodiments, the ultrahydrophilic PSA has a MVTR of at least about 2000 gram/m²/24 hours and a moisture uptake capacity of the adhesive layer of the device of at least about 1%, at least about 1.5%, at least about 2% or at least about 2.5% (all percentages by weight of the adhesive). In certain embodiments, the ultrahydrophilic PSA has a MVTR of at least about 2500 gram/m²/24 hours and a moisture uptake capacity of the adhesive layer of the device of at least about 1%, at least about 1.5%, at least about 2% or at least about 2.5% (all percentages by weight of the adhesive). In certain embodiments, the ultrahydrophilic PSA has a MVTR of at least about 3000 gram/m²/24 hours and a moisture uptake capacity of the adhesive layer of the device of at least about 1%, at least about 1.5%, at least about 2% or at. least about 2.5% (all percentages by weight of the adhesive). In certain embodiments the adhesive is an acrylic-based pressure sensitive adhesive. In certain embodiments the ultra-hydrophilic pressure-sensitive adhesive is DURO-TAK® 87-202A, or DURO-TAK® 80-222A (National Adhesives; New Jersey, USA).

In certain embodiments the cushioning device may be in the form of a finger cushion, a corn cushion, a bunion cushion, a callus cushion, and a plantar wart cushion. In certain embodiments the cushioning device will have a medicated layer coupled to the skin side of the cushioning layer. In certain embodiments, the medicated layer is between the cushioning layer and the adhesive layer, a medication in the medicated layer configured to pass through the adhesive layer to reach the skin when the device is applied. In certain embodiments, the adhesive layer is between the cushioning layer and the medicated layer, the adhesive layer extending outside a periphery of the medicated layer so that the adhesive extending outside the periphery of the medicated layer contacts the skin when the device is applied. In certain embodiments, the medicated layer further comprises keratolytic agent, for example salicylic acid.

In certain embodiments the cushioning layer may comprise a synthetic rubber foam, a woven cotton fabric, a synthetic fiber, a knit fabric, a silicone gel cushioning material, a polyurethane gel cushioning material, a polyurethane film, a polyvinyl chloride film, and/or a synthetic non-woven fabric.

The invention also provides a medical device for use on a finger or foot, comprising a medicated layer having a skin side and an outer side; an ultra-hydrophilic pressure-sensitive adhesive layer coupled to the skin side of the medicated layer.

The invention also provides a medical device for treating hyperkeratotic lesions comprising a cushioning means for providing cushioning of an area of the skin including the hyperkeratotic lesion; a medication means coupled to the cushioning means, the medication means for delivering a medication for treatment of hyperkeratotic lesions; and an ultra-hydrophilic pressure-sensitive adhesive coupled to the cushioning means and medication means. In certain embodiments, the hyper-keratotic lesion can be, for example, a corn or callus or a plantar wart. In certain embodiments the cushioning layer can be formed in the shape of a disk, a strip, or a bandage.

The invention also provides a method of treating skin diseases, comprising applying a medical device to an area of the finger or foot skin using an ultra-hydrophilic pressure-sensitive adhesive on the skin side of the device. In certain embodiments the method further comprises reducing hydration of the stratum corneum in the area by absorbing water in the medical device using an ultra-hydrophilic composition in the adhesive portion of the medical device. In certain embodiments, the method provides for a mean water vapor transfer through the adhesive layer of the device of at least about 1500 gram/m²/24 hours, at least about 2000 gram/m²/24 hours, at least about 2500 gram/m²/24 hours, or at least about 3000 gm/m²/24 hours. In certain embodiments the method provides for a moisture uptake capacity of the adhesive layer of the device of at least about 1%, at least about 1.5%, at least about 2% or at least about 2.5% (all percentages by weight of the adhesive). In certain embodiments, the method provides for a mean water vapor transfer through the adhesive layer of the device of at least about 1500 gram/m²/24 hours and provides for a moisture uptake capacity of the adhesive layer of the device of at least about 1% , at least about 1.5%, at least about 2% or at least about 2.5% (all percentages by weight of the adhesive). In certain embodiments, the method provides for a mean water vapor transfer through the adhesive layer of the device of at least about 2000 gram./m²/24 hours and provides for a moisture uptake capacity of the adhesive layer of the device of at least about 1% , at least about 1.5%, at least about 2% or at least about 2.5% (all percentages by weight of the adhesive). In certain embodiments, the method provides for a mean water vapor transfer through the adhesive layer of the device of at least about 2500 gram/m²/24 hours and provides for a moisture uptake capacity of the adhesive layer of the device of at least about 1% , at least about 1.5%, at least about 2% or at least about 2.5% (all percentages by weight of the adhesive). In certain embodiments, the method provides for a mean water vapor transfer through the adhesive layer of the device of at least about 3000 gram/m²/24 hours and provides for a moisture uptake capacity of the adhesive layer of the device of at least about 1% , at least about 1.5%, at least about 2% or at least about 2.5% (all percentages by weight of the adhesive).

The invention further provides a medical device for use on skin, for example on a hand or foot, comprising a medicated layer having a skin side and an outer side; an ultra-hydrophilic pressure-sensitive adhesive layer coupled to the skin side of the medicated layer; wherein the medical device provides a skin hold time that is greater than for a medical device comprising a hydrocolloid adhesive.

Further areas of applicability of the present invention will become apparent from the detailed description of some example embodiments provided hereinafter. It should be understood that the detailed description and specific examples, while describing some example embodiments of the invention, are intended for purposes of illustration only and are not intended to limit the scope of the invention.

### BRIEF DESCRIPTION OF THE DRAWINGS

The present invention will become more fully understood from the detailed description of example embodiments and the accompanying drawings, wherein:

FIG. 1a is a cross section view of an example cushioned medical device for use on a finger or foot, according to an example embodiment of this invention.

FIG. 1b illustrates an alternative configuration for the adhesive layer of the example cushioned medical device of FIG. 1.

FIG. 2 is a cross section view of another example cushioning device, according to an example embodiment of this invention.

FIG. 3a illustrates an uncushioned medical plaster sheet, according to an example embodiment of the present invention.

FIG. 3b illustrates a corresponding top view of FIG. 3a.

FIGS. 4a-4b are views of a moleskin cushion, according to an example embodiment of the present invention.

FIGS. 5a-5c are views of a bunion cushion, according to an example embodiment of the present invention.

FIGS. 6a-6d are views of a corn/callus cushion, according to an example embodiment of the present invention.

### DETAILED DESCRIPTION OF EXAMPLE EMBODIMENTS

The following description of some example embodiments is merely exemplary in nature and is in no way intended to limit the invention, its application, or uses.

Cells of the stratum corneum, the outermost layer of the epidermis, contain keratin, a protein that, together with the lipid component of the stratum corneum, helps keep the skin hydrated by slowing the rate of water evaporation. In addition, the cells of the stratum corneum can absorb water, aiding in skin hydration. However, these cells can also become highly hydrated, which results in wrinkling or maceration of the skin on the fingers and toes when the skin is immersed in water for prolonged periods.

Without wishing to be bound by theory, the inventors of the present application have observed that a device on a hand or foot is likely to be detached due to the cohesive failure within the weakened, hydrated stratum corneum layer of the skin rather than from an adhesive failure between the skin and the adhesive. Thus, the inventors believe it is advantageous to apply an adhesive that may reduce the hydration of the stratum corneum layer of the skin.

Some example embodiments of the present invention provide a cushioning device for use on a hand or foot. The cushioning device may include, on the skin side of the cushion, pressure-sensitive adhesive (“PSAs”) which are ultra-hydrophilic. These adhesives may provide an improved “stay on” effect, in part by reducing hydration of the stratum corneum. Other example embodiments of the present invention provide a medical device for use on a hand or foot, containing ultra-hydrophilic pressure-sensitive adhesives (“PSAs”) on the skin side of a medicated layer, e.g., a salicylic acid plaster for removal of plantar warts or corns, with a similar improved “stay on” effect. In some of the example embodiments of the present invention, the use of an ultra-hydrophilic pressure-sensitive adhesive may reduce the hydration of the stratum corneum layer of the skin on a hand or foot, which may, in turn, result in better adhesion, wearability and/or user comfort.

Another example embodiment of the present invention is a method of preventing or reducing stratum corneum hydration of a finger or foot by applying a medical device, e.g., a cushioning device or plaster, having an ultra-hydrophilic pressure-sensitive adhesive. In still another example embodiment, the present invention provides a method of treating finger or foot diseases by applying a cushioning device containing an ultra-hydrophilic pressure-sensitive adhesive.

Early skin adhesives (i.e., for bandages, wound covering, etc.) were primarily based on natural rubber based PSA formulations. Later, skin adhesives were based on synthetic rubber blend with tackifiers, plasticizers, oils, etc. More recently, the rubber based adhesives have been widely replaced by “acrylic” PSAs (e.g., typically copolymers of 2-ethylhexyl acrylate and acrylate/methacrylate monomers). In addition, the acrylic PSAs are typically more stable to UV and thermal aging. However, the acrylic PSAs are typically expensive.

More recently, a premium type of PSA for human skin applications has gained acceptance; these are the so-called “hydrocolloid” PSAs, as described in U.S. Pat. No. 6,656,495 to Cline et al. These hydrocolloid PSAs generally exhibit better adhesive wear performance than other PSA classes. They are usually produced by hot melt extrusion through dies and this limits their minimum thickness to about 0.01 to 0.03 inches, which is much thicker than typical solvent-based “acrylic” PSAs coated from solution. Yet greater thickness and the relatively slow extrusion process used to fabricate “hydrocolloid” PSAs tends to make them much less cost-effective than typical “acrylic” medical-grade skin PSAs.

In some of the example embodiments of the present invention, ultra-hydrophilic PSAs are used. A hydrophilic molecule (also called a polar molecule) or portion of a molecule is charge-polarized and capable of hydrogen bonding. Hydrophilic molecules generally dissolve or swell more readily in water than in oils. The ultrahydrophilic PSAs described in the present application have a significantly increased moisture vapor transmission rate (MVTR) and moisture uptake capacity (also referred to herein as “percent water uptake” or “moisture holding capacity”) as compared to conventional acrylic PSAs, generally without significantly decreasing the adhesion or other adhesive properties. In separate example embodiments, commercially available ultrahydrophilic acrylic PSAs supplied by National Starch & Chemical (Duro-Tak 87-202A and Duro-Tak 80-222A) have an MVTR of 3250 gram/m²/24 hours and a percent water uptake value of 2.66% w/w. In comparison, a conventional medical grade acrylic PSA (Duro-Tak 80-1197) has a MVTR of 1207 gram/m²/24 hours and percent water uptake value of 0.8% w/w.

The ultra-hydrophilic PSAs in the example embodiments of the present invention are expected to greatly reduce the hydration level of the stratum corneum of a human being without the adhesive losing significant internal cohesive strength. The ultra-hydrophilic PSAs have a high MVTR, which is about three times higher than conventional medical grade acrylic skin adhesives. The ultra-hydrophilic PSAs also have a high level of moisture holding capacity, which is also about three times higher than conventional medical grade acrylic skin adhesives. The conventional adhesive indicated above, for example, is an early generation conventional acrylic-based skin adhesive. Thus, in certain example embodiments of the invention a medical device is provided that provides a skin hold time that is greater than for a similar medical device comprising a hydrocolloid adhesive or an early generation conventional acrylic-based skin adhesive. As used herein “skin hold time” refers to the amount of time a medical device stays on the skin during normal usage. In certain embodiments, the skin hold time will be at least 24 hours, at least 48 hours and at least 72 hours and greater.

Suitable ultra-hydrophilic PSA adhesive material for the examples given below are sold by National Adhesives, Bridgewater, N.J. under the designation, DURO-TAK® 87-202A and DURO-TAK® 80-222A, noted above. The adhesive is an acrylic, self-curing, pressure-sensitive adhesive which is supplied by the manufacturer in an organic solvent solution. It will be appreciated that other adhesives that exhibit ultra-hydrophilic properties may also be employed, including other acrylic or acrylic-rubber hybrid adhesives that exhibit ultra-hydrophilic properties. As used herein, “acrylic-based pressure sensitive adhesives” refer to a class of acrylic based adhesives that exclude those adhesives described herein as “hydrocolloid” adhesives.

Some particular example embodiments are discussed below. In the present application, coupled is used to mean connected in any manner, including, e.g., integrated in a single component, attached in direct contact, bonded adhesively, or mechanically connected indirectly through one or more other components. In the examples and drawings described below, similar elements in different examples are shown with the same number and using primes to denote that the elements may be made from similar materials and have substantially similar functions, but may have slightly different configurations and dimensions in different embodiments. Cushions are commonly made from one or more layers of woven cotton cloth (sometimes referred to as “moleskin”), typically approximately 0.04-0.05 inches in thickness; from synthetic rubber foam (sometimes referred to as “peachfoam”), typically approximately 0.13-0.15 inches in thickness; from low density polyethylene foam, typically approximately 0.016 to approximately 0.125 inches in thickness; and from polyurethane molded gels, typically approximately 0.02 to approximately 0.04 inches in thickness.

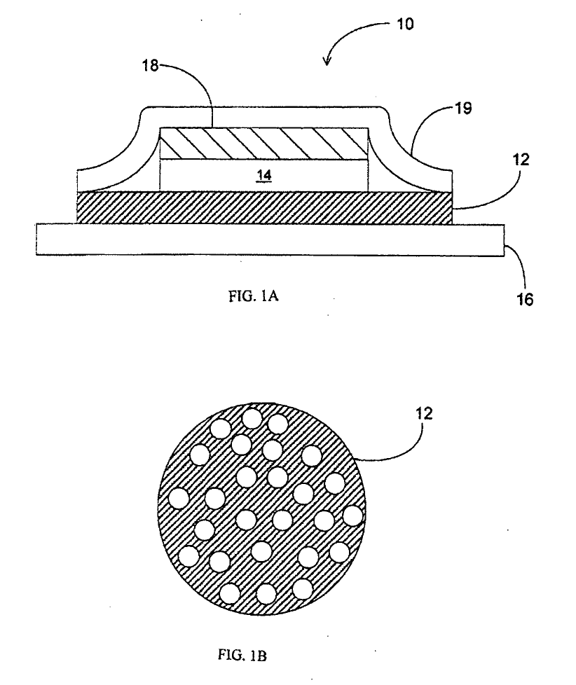

FIG. 1a is a cross section view of an example cushioned medical device for use on a finger or foot, according to an example embodiment of this invention. The device 10 may be used, for example, for removing corns, calluses and warts. Device 10 may include an adhesive layer 12 of a material having adhesive and moisture absorbing and transmitting qualities. In this regard, a preferred material of layer 12 may be the ultra-hydrophilic adhesive material described previously. When used as a corn remover, layer 12 may have the shape of a rhombus with a greatest length of about 1.375 inches and a greatest width of about 0.50 inch. However, ultra-hydrophilic adhesive layer 12 may have different configurations and dimensions depending upon the particular application, and the specific shape and dimensions are not relevant to the present invention. Ultra-hydrophilic adhesive layer 12 provides greater adhesive properties in moisture conditions, e.g., the environment found inside a shoe or other footwear. Thus, when placed on a person's skin, ultra-hydrophilic adhesive layer 12 will stick to the person's skin, and as the person's skin moisture increases, the ultra-hydrophilic adhesive layer 12 will not lose its adhesive activity. It will therefore be appreciated that in the presence of a liquid and/or moisture, the ultra-hydrophilic layer should help to prevent maceration and weakening of the mechanical strength of the stratum corneum, with the result that the adhesive holding power of the device 10 does not decrease as rapidly as would occur with a conventional acrylic PSA. This means that device 10 should adhere to a person's skin for a longer period of time than cushions or medicated pads using a conventional acrylic PSA.

Coupled to the adhesive layer 12 may be a medicated layer 14. The medicated layer may contain an active medical ingredient in various forms, e.g., a solid, gel, cream, or plaster. One example for the treatment of hyperkeratotic skin lesions would be a salicylic acid plaster formulation containing about 40% salicylic acid by weight. Other skin pads might contain moisturizing formulations, with salicylic acid, lactic acid, urea, glycerol, etc. and/or cooling formulations containing menthol, methyl salicylate, etc. In the example shown, the medicated layer may be in direct contact with the adhesive layer 12, which may be applied directly to the medicated layer. In an alternative example embodiment, the adhesive and and the medication layer may be indirectly coupled through additional layers. In a further alternative example embodiment, the adhesive layer and medication layer may be coupled by being integrated into a single homogenous layer containing both the medication and the adhesive.

In the example device 10, the adhesive layer 12 extends beyond the boundaries of the medicated layer 14 and lies completely between the skin and the medicated layer when the device 10 is applied. This configuration is suitable for when the medication is of a type that readily passes through the adhesive layer. Alternatively, if the adhesive is not permeable to the particular medication, the adhesive layer may be applied discontinuously, e.g., with holes or openings, so that the medication can pass through the adhesive layer, as shown in FIG. 1b. In this case, the rate of the dispersion of the medication from the medicated layer 14 may be controlled, at least in part by the size and number of holes provided in the adhesive layer. In an alternative example embodiment, the cushion and the medicated layer may be integrated, e.g., by dispersing the medication in a solvent in the cushioning layer.

When manufactured or distributed, the device 10 may also include a removable release liner 16, which may cover the adhesive layer completely prior to application of the device 10. The release layer may he coated paper, polyethylene coated paper, or a plastic film (e.g., polyethylene terephthalate), all of which would be coated with a very thin layer of silicone release agent, or other material that does not bond strongly to the adhesive layer. In use, a person pulls up the portion of release liner 16 to remove device 10 from release liner 16. The device 10 is then placed on the person's skin with the medicated layer immediately above the area of the skin to be treated, e.g., corn, callus or wart to be removed. Adhesive layer 12 functions to secure device 10 thereon. Further measures may be taken to secure the device 10.

The example device may also include a cushioning layer 18 which may be, e.g., a synthetic rubber foam, a woven cotton fabric, a synthetic fiber, a knit fabric, a silicone gel cushioning material, a polyurethane gel cushioning material, a polyurethane film, a polyvinyl chloride film, or a synthetic non-woven fabric.

The example device may also have a barrier layer 19 which may be. provided over the medication and adhesive layer opposite from the release layer. The barrier layer may be moisture permeable or impermeable depending on the particular application. The barrier layer may also he in place to impart stability to the device, for example by protecting the medications from water.

In example device 10, the adhesive layer 12 extends beyond the boundaries of the medicated layer 14 and to the periphery of barrier layer 19, insuring that the outer edges of the barrier layer firmly bond to the skin. In an alternative example, the periphery of all the various layers could be aligned.

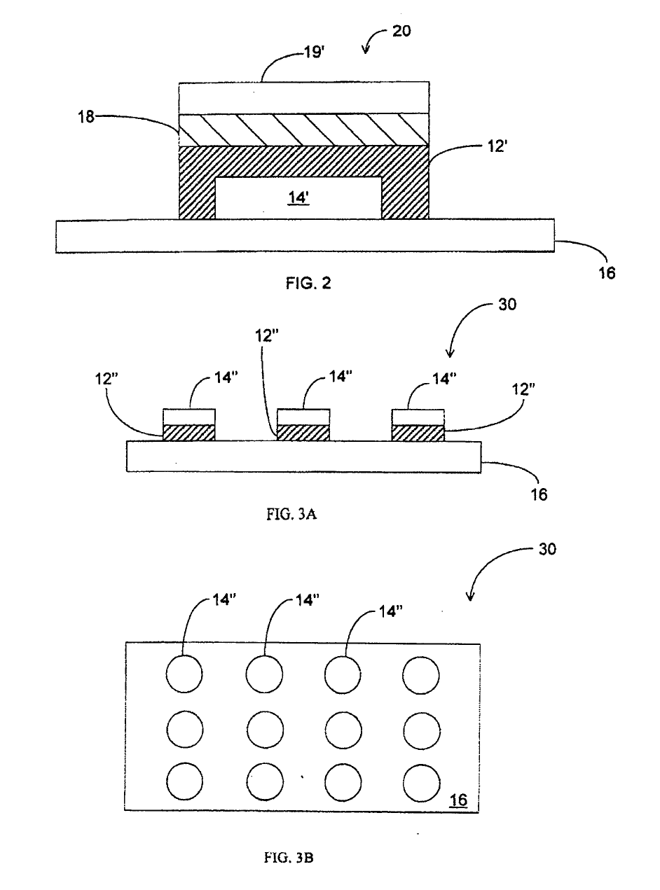

FIG. 2 is a cross section view of another example medical cushioning device for use on a finger or foot, according to an example embodiment of this invention. In example cushioning device 20, the medicated layer 14′, similar in composition to the medicated layer in 14 shown in the previous example, may be positioned on the skin side of the device. The hydrophilic adhesive layer 12′ may be positioned above and surrounding the medicated layer 14′. It will be appreciated that depending on the manufacturing process, the adhesive may be omitted from all or part of the area above the medicated layer, e.g., by use of an adhesive doughnut shape surrounding the outer edges of adhesive layer. The release liner 16 covers the skin side of both the adhesive layer and the medicated layer. Cushioning layers 18′ and outer barrier layer 19′ may also be provided similarly to the corresponding layers described in the previous example.

In example device 20, the underside of ultra-hydrophilic adhesive layer 12′ can be provided with a recess at the center thereof, and medicated layer 14′ in spaced relation to the side walls of recess. In such case, a portion of medicated layer 14′ may extend out from recess to a lower height than the lower surface of ultra-hydrophilic adhesive layer 12.

FIGS. 3a and 3b illustrate, respectively, a cross section and a top view of an uncushioned medical plaster sheet, according to an example embodiment of the present invention. The example sheet 30 may include a plurality of medicated plaster layers 14″ attached by ultra-hydrophilic adhesive layers 12″ to a release liner 16. This configuration allows multiple plasters to be supplied for treatment of conditions over the course of an extended period of time.

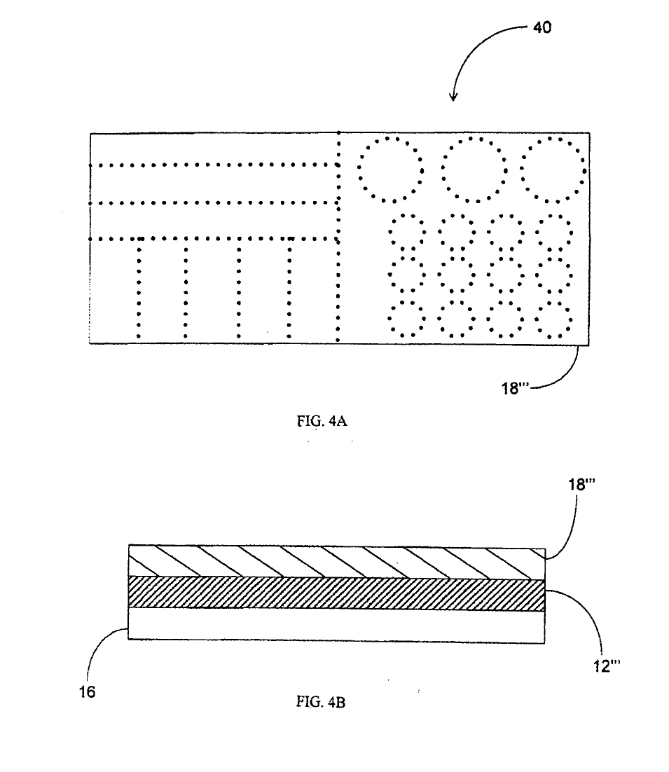

FIGS. 4a and 4b illustrate an unmedicated cushion, according to an example embodiment of the present invention. FIG. 4a is a top view of an example of a moleskin cushion 40 and FIG. 4b is a corresponding cross section of cushion 40. As shown in FIG. 4a, the example cushion may be perforated or die cut in a variety of shapes and configured to allow a user to easily detach a piece of a desired size and shape. The adhesive layer 12′″ may be covered by a cushioning layer 18′″. It will be appreciated that the moleskin cushion 40 can be further cut into sizes suitable to certain area of a foot or finger, e.g., to prevent blisters or chafing, or to reduce the discomfort and promote the healing of blisters or chafing which has already occurred. In an alternative embodiment, smaller pre-cut cushions can be provided on the release sheet 16 in any of a variety of shapes, e.g., circles, squares, doughnut shaped rings, etc. It will be appreciated that medicated versions of the example cushion may also be provided. It will also be appreciated that multi-layer cushions may also be provided, for example with one layer to provide cushioning and another smoother layer on the top to reduce friction.

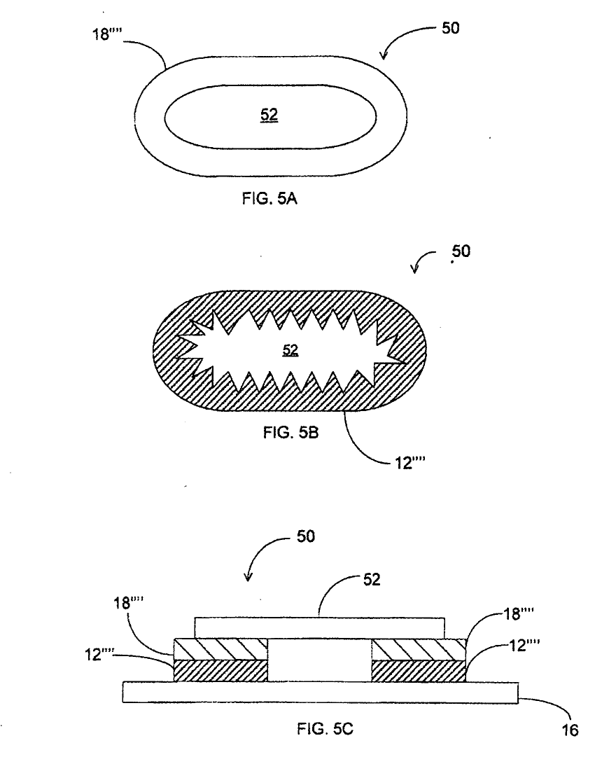

FIGS. 5a-5c illustrate a non-medicated bunion cushion, according to an example embodiment of the present invention. FIG. 5a and FIG. 5b are corresponding bottom and top views cushion 50, while FIG. 5c presents a cross-sectional view of cushion 50. As illustrated in FIG. 5c, example bunion cushion 50 may include a doughnut-shaped cushioning layer 18″″ over a similarly shaped ultra-hydrophilic adhesive layer 12″″. Most of (or alternatively the entire) doughnut-shaped structure may be covered by an upper layer 52, which may simply cover the device, or may also include additional cushioning. The example cushion 50 may be provided on a release sheet 16, e.g., with multiple cushions provided on a single sheet.

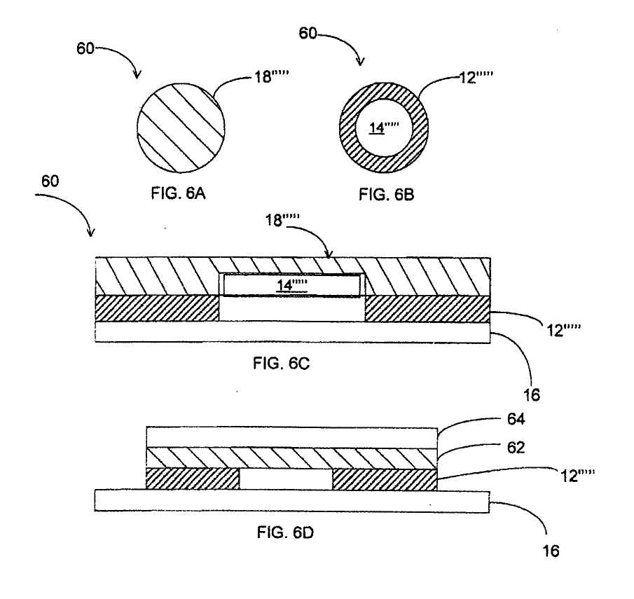

FIGS. 6a-6d illustrate a corn/callus plaster, according to an example embodiment of the present invention. Similar devices may he used for treating other types of hyper-keratotic lesions. FIG. 6a shows a top view of example plaster 60, while FIG. 6b shows a bottom view and FIG. 6c shows a cross-sectional view. As illustrated in FIG. 6c, a corn cushion 60 be provided on a releasable sheet 16, an adhesive layer 12′″″ positioned on the periphery of a cushioning layer 18″″. It will be appreciated that multiple example plasters may be provided on a single release sheet. It will also be appreciated that, although the example device 60 is illustrated as a circular disk, it may be provided in a variety of other shapes, e.g., rectangular strips, as part of a bandage, or in a large rectangular sheet that may be cut to shape. In the example plaster, a medicated layer 14′″″ may be positioned in the center of the cushioning layer 18″″. In an alternative structure shown in FIG. 6d, the cushioning and medication layer may be integrated in layer 62 and covered by an occlusive barrier layer 64 that prevents the spread of the medication from the medication layer to the surrounding skin area.

Yet another example embodiment of the present invention is a method of treating skin diseases, e.g., hyper-keratotic lesions of the foot or finger, such as corns, calluses, and plantar warts, by applying any of the cushioning devices described above on an area of a finger or foot skin, so that a medicament can be topically dispersed on or into the skin.

Although some of the example embodiments of the present invention have been discussed in relation to devices for removing warts, corns and calluses and containing salicylic acid as the keratolytic agent, it will be appreciated that any other keratolytic agent and/or medicament, such as an antibiotic agent, antimicrobial agent, antifungal agent or the like may also be used.

Another advantage that may be obtained with the example embodiments described above is that less medicament will be lost to the environment. This is due to the combination of ultra-hydrophilic adhesive layer with medicated layers. As a result, the devices may be used longer than conventional devices and will hold more securely.

Having described specific preferred embodiments of the invention with reference to the accompanying drawings, it will be appreciated that the present invention is not limited to those precise embodiments and that various changes and modifications can be effected therein by one of ordinary skill in the art without departing from the scope or spirit of the invention as defined by the appended claims.

What is claimed is:

1. A medical cushioning device for use on a finger or foot, comprising:
a cushioning layer having a skin side and an outer side; and
an ultra-hydrophilic pressure-sensitive adhesive layer coupled to the skin side of the cushioning layer.

2. The cushioning device of claim 1, wherein the ultra-hydrophilic pressure-sensitive adhesive layer is in direct contact with the cushioning layer.

3. The cushioning device of claim 1, wherein the pressure sensitive adhesive has a moisture vapor permeability of at least about 1500 gm/m²/24 hours.

4. The cushioning device of claim 1, wherein the pressure sensitive adhesive has a moisture vapor permeability of at least about 2000 gm/m²/24 hours.

5. The cushioning device of claim 1, wherein the pressure sensitive adhesive has a moisture vapor permeability of at least about 2500 gm/m²/24 hours.

6. The cushioning device of claim 1, wherein the pressure sensitive adhesive has a moisture vapor permeability of at least about 3000 gm/m²/24 hours.

7. The cushioning device of claim 1, wherein the pressure sensitive adhesive has a moisture uptake capacity of at least about 1% by weight of the adhesive.

8. The cushioning device of claim 1, wherein the pressure sensitive adhesive has a moisture uptake capacity of at least about 1.5% by weight of the adhesive.

9. The cushioning device of claim 1, wherein the pressure sensitive adhesive has a moisture uptake capacity of at least about 2% by weight of the adhesive.

10. The cushioning device of claim 1, wherein the pressure sensitive adhesive has a moisture uptake capacity of at least about 2.5% by weight of the adhesive.

11. The cushioning device of claim 1, wherein the adhesive is an acrylic-based pressure sensitive adhesive.

12. The cushioning device of claim 1, wherein the adhesive is an acrylic-rubber hybrid adhesive.

13. The cushioning device of claim. 1, wherein the cushioning device is selected from a group consisting of a finger cushion, a hand cushion, a toe cushion and a foot cushion.

14. The cushioning device of claim 13 wherein the cushioning device comprises a foot cushion chosen from the group consisting of a corn cushion, a bunion cushion, a callus cushion, and a plantar wart cushion.

15. The cushioning device of claim 1, further comprising:
a medicated layer coupled to the skin side of the cushioning layer.

16. The cushioning device of claim 15, wherein the medicated layer is between the cushioning layer and the adhesive layer, a medication in the medicated layer configured to pass through the adhesive layer to reach the skin when the device is applied.

17. The cushioning device of claim 15, wherein the adhesive layer is between the cushioning layer and the medicated layer, the adhesive layer extending outside a periphery of the medicated layer so that the adhesive extending outside the periphery of the medicated layer contacts the skin when the device is applied.

18. The cushioning device of claim 15, wherein the medicated layer further comprises a keratolytic agent.

19. The cushioning device of claim 18, wherein the keratolytic agent further comprises salicylic acid.

20. The cushioning device of claim 1, wherein the ultra-hydrophilic pressure-sensitive adhesive is DURO-TAK® 87-202A.

21. The cushioning device of claim 1, wherein the ultra-hydrophilic pressure-sensitive adhesive is DURO-TAK® 80-222A.

22. The device of claim 1, wherein the cushioning layer is selected from a group comprising a synthetic rubber foam, a woven cotton fabric, a synthetic fiber, a knit fabric, a silicone gel cushioning material, a polyurethane gel cushioning material, a polyurethane film, a polyvinyl chloride film, and a synthetic non-woven fabric.

23. The device of claim 1, further comprising: a barrier layer positioned above the medicated layer.

24. The device of claim 1, wherein another material layer may be added to the skin side of the cushioning layer.

25. A medical device for use on a finger, hand, toe, or foot, comprising:
a medicated layer having a skin side and an outer side;
an ultra-hydrophilic pressure-sensitive adhesive layer coupled to the skin side of the medicated layer.

26. The device of claim 25, further comprising:
a coating layer coupled to the outer side of the medicated layer.

27. The medical device of claim 25, further comprising:
a cushioning layer coupled to the outer side of the medicated layer.

28. The device of claim 25, wherein the medicated layer further comprises a keratolytic agent.

29. The cushioning device of claim 28, wherein the keratolytic agent further comprises salicylic acid.

30. A medical device for treating hyperkeratotic lesions comprising:
a cushioning means for providing cushioning of an area of the skin including the hyperkeratotic lesion;
a medication means coupled to the cushioning means, the medication means for delivering a medication for treatment of hyperkeratotic lesions; and
an ultra-hydrophilic pressure-sensitive adhesive coupled to the cushioning means and medication means.

31. The medical device of claim 30, wherein the medication means further comprises a keratolytic agent.

32. The cushioning device of claim 31, wherein the keratolytic agent further comprises salicylic acid.

33. The medical device of claim 32, wherein the salicylic acid is about 40% by weight of a medicated material in the medication means.

34. The medical device of claim 30, wherein the hyper-keratotic lesion is a corn or callus.

35. The medical device of claim 30, wherein the hyper-keratotic lesion is a plantar wart.

36. The medical device of claim 30, wherein the cushioning layer is in a shape of a disk, a strip, or a bandage.

37. A method of treating skin diseases, comprising:
applying a medical device to an area of the finger or foot skin using an ultra-hydrophilic pressure-sensitive adhesive on the skin side of the device.

38. The method of claim 37, wherein the skin disease is a hyperkerotic skin condition and the medical device includes a keratolytic agent.

39. The method of claim 38, wherein the keratolytic agent is salicylic acid.

40. The method of claim 37, further comprising:
reducing hydration of the stratum corneum in the area by absorbing water in the medical device using an ultra-hydrophilic composition in the adhesive portion of the medical device.

41. The method of claim 37, further comprising:
reducing hydration of the stratum corneum in the area by providing a mean water vapor transfer through the adhesive layer of the device of at least 1500 gm/m²/24 hours.

42. The method of claim 37, further comprising:
reducing hydration of the stratum corneum in the area by providing a mean water vapor transfer through the adhesive layer of the device of at least 2000 gm/m²/24 hours.

43. The method of claim 37, further comprising:
reducing hydration of the stratum corneum in the area by providing a mean water vapor transfer through the adhesive layer of the device of at least 2500 gm²/24 hours.

44. The method of claim 37, further comprising:
reducing hydration of the stratum corneum in the area by providing a mean water vapor transfer through the adhesive layer of the device of at least 3000 gm/m²/24 hours.

45. The method of claim 37, further comprising:
reducing hydration of the stratum corneum in the area by providing a moisture uptake capacity of the adhesive layer of at least about 1% by weight of the adhesive.

46. The method of claim 37, further comprising:
reducing hydration of the stratum corneum in the area by providing a moisture uptake capacity of the adhesive layer of at least about 1.5% by weight of the adhesive.

47. The method of claim 37, further comprising:
reducing hydration of the stratum corneum in the area by providing a moisture uptake capacity of the adhesive layer of at least about 2% by weight of the adhesive.

48. The method of claim 37, further comprising:
reducing hydration of the stratum corneum in the area by providing a moisture uptake capacity of the adhesive layer of at least about 2.5% by weight of the adhesive.

49. A medical device for use on skin, comprising:
a medicated layer having a skin side and an outer side;
an ultra-hydrophilic pressure-sensitive adhesive layer coupled to the skin side of the medicated layer;
wherein the medical device provides a skin hold time that is greater than for a medical device comprising a hydrocolloid adhesive.

50. The medical device of claim 49, further comprising:
a coating layer coupled to the outer side of the medicated layer.

51. The medical device of claim 49, further comprising:
a cushioning layer coupled to the outer side of the medicated layer.

52. The medical device of claim 49, wherein the medicated layer further comprises keratolytic agent.

53. The medical device of claim 52, wherein the keratolytic agent further comprises salicylic acid.
